(19) United States
(12) Patent Application Publication
(10) Pub. No.: US 2024/0307052 A1
(43) Pub. Date: Sep. 19, 2024

(54) MEDICAL SYSTEMS, DEVICES, AND RELATED METHODS FOR LIFTING TISSUE

(71) Applicant: Boston Scientific Scimed, Inc., Maple Grove, MN (US)

(72) Inventors: Yeison CALVO, San Antonio (CR); Barry WEITZNER, Acton, MA (US); Juan Pablo ORTIZ GARCIA, Heredia (CR); Gian Franco LOO FUCHS, San Jose (CR); Jimena CESPEDES BERROCAL, Alajuela (CR); Cristian ARAYA CAMACHO, Tambor de Alajuela (CR); Julian FUENTES CASTRO, La Unión (CR); Pablo MONTERO RODRIGUEZ, La Unión (CR)

(73) Assignee: Boston Scientific Scimed, Inc., Maple Grove, MN (US)

(21) Appl. No.: 18/604,600

(22) Filed: Mar. 14, 2024

Related U.S. Application Data
(60) Provisional application No. 63/490,259, filed on Mar. 15, 2023.

(51) Int. Cl. A61B 17/04; A61B 1/018; A61B 17/00; A61B 17/06; A61B 17/128

(52) U.S. Cl. CPC A61B 17/0469; A61B 1/018; A61B 17/1285; A61B 2017/0034; A61B 2017/00473; A61B 2017/00818; A61B 2017/00876; A61B 2017/00951; A61B 2017/0409; A61B 2017/0441; A61B 2017/0618

(57) ABSTRACT

A medical system includes an insertion device having a working channel, a first medical device configured to be disposed within the working channel of the insertion device, and a second medical device configured to be disposed within the working channel of the insertion device. The first medical device includes a handle, a control element extending from the handle, a detachable portion, a helical coil coupled to the detachable portion, and a loop. The detachable portion is removably coupled to a distal portion of the control element. A first portion of the loop is coupled to the detachable portion, and a second portion of the loop extends radially away from the detachable portion. The second medical device is configured to receive the second portion of the loop. The second medical device is configured to couple the second portion of the loop to a portion of a treatment site.

### CROSS-REFERENCE TO RELATED APPLICATIONS

This application claims the benefit of priority of U.S. Provisional Patent Application No. 63/490,259, filed Mar. 15, 2023, the entirety of which is incorporated herein by reference.

### TECHNICAL FIELD

This disclosure generally relates to medical systems, devices, and related methods that may be used to treat a subject. More particularly, at least some embodiments of the present disclosure relate to systems, devices, and related methods for lifting tissue.

### BACKGROUND

Organ walls are often composed of several layers: the mucosa (the surface layer), the submucosa, the muscularis (muscle layer), and the serosa (connective tissue layer). In gastrointestinal, colonic, and esophageal cancer, lesions or cancerous masses may form along the mucosa and often extend into the lumens of the organs. Conventionally, the condition is treated by cutting out a portion of the affected organ wall. This procedure, however, may cause discomfort to patients, and/or pose health risks.

Physicians have adopted minimally invasive techniques, such as endoscopic procedures like endoscopic mucosal resection (EMR) and endoscopic submucosal dissection (ESD). EMR methods are typically used for removal of small cancerous or abnormal tissues (e.g., polyps), and ESD methods are typically used for en bloc removal of large cancerous or abnormal tissues (e.g., lesions). These procedures are generally performed with an endoscope and other instruments. During these procedures, the endoscope may be passed through a percutaneous incision, passed down the throat, or guided through the rectum to reach tissue targeted for resection or dissection, such tissue having an abnormality such as a lesion or cancerous mass in an affected organ. The abnormality is generally identified and marked.

The mucosal layer containing the abnormality is then separated from the underlying tissue layers using a medical instrument extending through a working channel, or a lumen, of the endoscope. The abnormality is subsequently removed using the same or different medical instrument. Conventionally, tissue is removed by employing a cutting device such as a wire loop or knife, which may be adapted for electrocautery. Subsequently, excised tissue may be extracted for examination or disposal using a tissue removal device, such as a grasper or other device for holding tissue. Both the cutting device and tissue removal device often need to apply a large traction force or lifting force on the tissue during the procedure, and such a lifting force may be limited by conventional devices. The limited lifting force in conventional tissue cutting and grasping devices may cause increased procedure time, patient injury, or other procedural complications.

The systems, devices, and methods of this disclosure may rectify some of the deficiencies described above or address other aspects of the art.

### SUMMARY

Examples of this disclosure relate to, among other things, systems, devices, and methods for performing one or more medical procedures with the medical devices. Specifically, this disclosure includes medical systems and devices comprising an end effector and methods of use thereof (e.g., methods of delivering the end effector to a treatment site of a patient, for example, to lift tissue). Each of the examples disclosed herein may include one or more of the features described in connection with any of the other disclosed examples.

According to one aspect, a medical system may comprise an insertion device having a working channel, a first medical device, and a second medical device. The first medical device may be configured to be disposed within the working channel of the insertion device. The first medical device may have a handle, a control element extending from the handle, a detachable portion, a helical coil coupled to the detachable portion, and a loop. The detachable portion may be removably coupled to a distal portion of the control element. The helical coil may be coupled to the detachable portion. The loop may include a first portion and a second portion. The first portion of the loop may be coupled to the detachable portion. The second portion of the loop may extend radially away from the detachable portion. The second medical device may be configured to be disposed within the working channel of the insertion device. The second medical device may be configured to receive the second portion of the loop. The second medical device may be configured to couple the second portion of the loop to a portion of a treatment site.

The medical system may include one or more of the following features. The first medical device may further include a coupler. The coupler may include a first portion coupled to or part of the distal portion of the control element. The coupler may include a second portion coupled to or part of the proximal portion of the detachable portion. The first portion and the second portion may be configured to releasably couple the detachable portion to the control element. The first portion of the coupler may be configured to overlap with at least a portion of the second portion of the coupler.

In some configurations, a first contacting surface of the first portion may abut a second contacting surface of the second portion. The first contacting surface of the first portion may be complementarily shaped to the contacting surface of the second portion. In some embodiments, a portion of the first portion of the coupler may be magnetic. A portion of the second portion of the coupler may be magnetic of an opposite polarity of the portion of the first portion.

A portion of the first portion of the coupler may include an electromagnet, and a portion of the second portion of the coupler may be a magnetic material in some embodiments.

In some configurations, rotation of the helical coil in a first direction may be configured to couple the distal portion to a tissue wall. Rotation of the helical coil in a second direction may be configured to uncouple the helical coil from the tissue wall. The helical coil may include a plurality of windings. Adjacent windings of a first portion of the plurality of windings may be spaced by a first distance. Adjacent windings of a second portion of the plurality of windings may be spaced by a second distance. Adjacent windings of a third portion of the plurality of windings may be spaced by a third distance. In some embodiments, the third distance may be greater than the second distance, and the second distance may be greater than the first distance.

In some embodiments, the handle may include a stationary body, a first movable body, and a second movable body. The first movable body may control axial movement of the distal portion of the control element. The second movable body may control rotational movement of the distal portion of the control element. The stationary body may include a first indicator to identify axial movement of the helical coil. The second movable body may include a second indicator to identify the rotational movement of the helical coil.

In some configurations, the second medical device may include a clip. The second portion of the loop may be comprised of a resilient material in some configurations. The medical system may further include a third medical device configured to be disposed within the working channel of the insertion device in some configurations. The third medical device may be configured to cut tissue.

In some configurations, the first portion of the coupler and the second portion of the coupler may be coupled by an adhesive material. In a first configuration, the material may be disposed within a sheath extending from the handle. In a second configuration, the material may be uncovered by the sheath. In the second configuration, the material may degrade to uncouple the first portion of the coupler and the second portion of the coupler.

According to an alternative embodiment, a medical device may include a handle, a control element, a detachable portion, a helical coil, and a loop. The control element may extend from the handle. The detachable portion may be removably coupled to a distal portion of the control element. The helical coil may be coupled to the detachable portion. The loop may have a first portion and a second portion. The first portion of the loop may be coupled to the detachable portion. The second portion of the loop may extend radially outward from the detachable portion.

The medical device may include one or more of the following features. In some configurations, the first medical device may further include a coupler. The coupler may include a first portion coupled to or part of the distal portion of the control element. The coupler may include a second portion coupled to or part of the proximal portion of the detachable portion. The first portion and the second portion may be configured to releasably couple the detachable portion to the control element. A portion of the first portion of the coupler may be a magnet, and a portion of the second portion of the coupler may be me a magnetic material.

According to another aspect, a method may include delivering an insertion device and a first medical device into a lumen to a treatment site. The first medical device may be configured to be disposed within a working channel of the insertion device. The first medical device may include a handle, a control element, a detachable portion, a helical coil, and a loop. The control element may extend from the handle. The detachable portion may be removably coupled to a distal portion of the control element. The helical coil may be coupled to the detachable portion. A first portion of the loop may be coupled to the detachable portion, and a second medical device may be configured to receive a second portion of the loop.

The method may further include coupling the helical coil to a wall of the lumen, uncoupling the detachable portion from the control element, and delivering a second medical device. The second medical device may be configured to be disposed within the working channel of the insertion device. The second medical device may include a detachable clip. The method may further include attaching the detachable clip to the loop, coupling the detachable clip to the treatment site, delivering a third medical device, and resecting at least a portion of the treatment site using the third medical device. The third medical device may be configured to be disposed within the working channel of the insertion device.

After delivering the third medical device and partially resecting the treatment site with the third medical device, the method may further include delivering the first medical device, coupling the first medical device to the detachable portion, including the helical coil, and repositioning the detachable portion. The method may further include delivering the third medical device and resecting at least a second portion of the treatment site using the third medical device.

Any of the examples described herein may have any of these features in any combination.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1A illustrates a perspective view of a medical device, including a distal portion, in a first or coupled configuration, and FIG. 1B illustrates a perspective view of the distal portion of the medical device in a second configuration, according to aspects of this disclosure.

FIGS. 2A-2E illustrate perspective views of a distal portion of an exemplary medical system at various states during operation, according to aspects of this disclosure.

FIG. 3 is a flow diagram of an exemplary method, according to aspects of this disclosure.

### DETAILED DESCRIPTION

Reference will now be made in detail to aspects of the present disclosure, examples of which are illustrated in the accompanying drawings. Wherever possible, the same or similar reference numbers will be used through the drawings to refer to the same or like parts. The term “distal” refers to a portion farthest away from a user when introducing a device into a subject (e.g., patient). By contrast, the term “proximal” refers to a portion closest to the user when placing the device into the subject.

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of +10% in a stated value or characteristic.

Although the treatment site is discussed herein as being in the subject's gastrointestinal tract, this disclosure is not so limited, as the treatment site may be any internal lumen, organ, cavity, or other tissue within the subject. Additionally, although endoscopes are referenced herein, it will be appreciated that the disclosure encompasses any medical devices having a working channel, or a lumen, extending from a proximal end to a distal end, such as ureteroscopes, duodenoscopes, gastroscopes, endoscopic ultrasonography (“EUS”) scopes, colonoscopes, bronchoscopes, laparoscopes, arthroscopes, cystoscopes, aspiration scopes, sheaths, or catheters.

Embodiments of the disclosure may address one or more of the limitations in the art. The scope of the disclosure, however, is defined by the attached claims and not the ability to solve a specific problem. The present disclosure is drawn to systems, devices, and related methods, for a tissue lifting medical device, among other aspects.

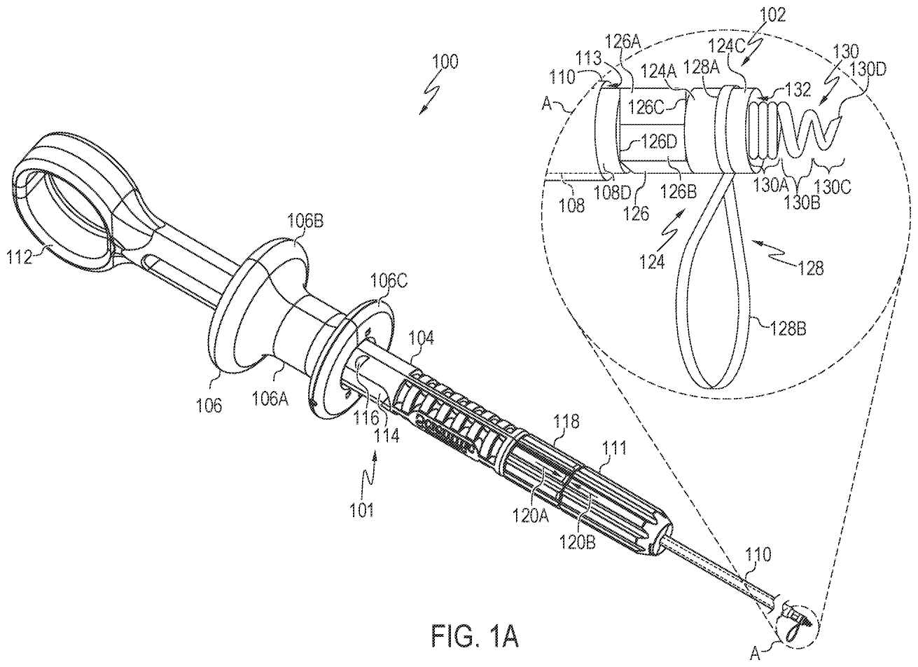

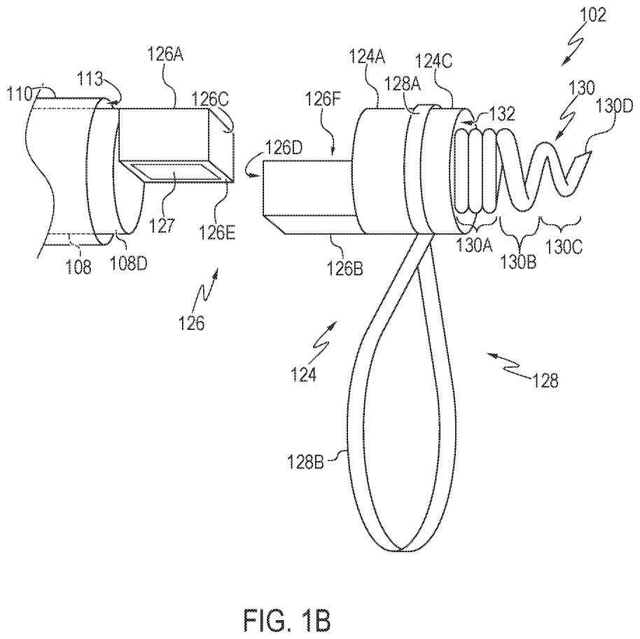

FIG. 1A shows a perspective view of a medical device 100 having a handle 101 and a distal portion 102 in a first (e.g., coupled or attached) configuration. FIG. 1B illustrates distal portion 102 of medical device 100 in a second (e.g., uncoupled or detached) configuration. One or more portions of handle 101 may be movable or manipulatable to control various aspects of distal portion 102. One or more portion(s) of distal portion 102 may be movable to facilitate, for example, coupling and/or uncoupling distal portion 102 to tissue and/or uncoupling distal portion 102 from medical device 100. Distal portion 102 is shown in detail in circle A of FIG. 1A.

Referring to FIG. 1A, handle 101 may include a stationary body 104 and a first movable body, or a first actuator, 106. Movement of first movable body 106 relative to stationary body 104 may control the movement (e.g., extension or retraction) of a portion of distal portion 102. For example, one or more control elements 108 may be coupled to first movable body 106 and to a portion of distal portion 102, and thus first movable body 106 may control the movement (e.g., axial movement, such as extension or retraction) of one or more portions of distal portion 102. Control element(s) 108 may be comprised of any rigid or semi-rigid material, such as, for example, metals, polymers, metal-polymer composites, and the like. Control element(s) 108 may be formed by a coil, a braid, a tube, a sheath, etc. Control element(s) 108 may be coupled (e.g., directly or indirectly) or otherwise connected to first movable body 106 via, for example, an adhesive, a mechanical fit, a snap fit, a press fit, a weld (e.g., a laser weld), or any other coupling techniques commonly known in the art. In some embodiments, multiple control elements 108 are used. In such embodiments, each of the multiple control elements 108 are formed of the same or different materials and/or coupled or otherwise connected to distal portion 102 and first movable body 106 via the same or different coupling techniques.

An external sheath 110 may be coupled (e.g., directly or indirectly) or otherwise connected to a stationary distal handle portion 111 of handle 101, for example, an adhesive, a mechanical fit, a snap fit, a press fit, a weld (e.g., a laser weld), or any other coupling techniques commonly known in the art. Control element(s) 108 may be movable within external sheath 110, for example, within a lumen 113. A proximal portion of stationary body 104 may include a finger ring 112, for example, to receive the user's thumb.

First movable body 106 may at least partially surround a portion of stationary body 104, and a portion of first movable body 106 may at least partially extend into a handle slot 114. Handle slot 114 may extend longitudinally through at least a portion of stationary body 104 of handle 101. In some aspects, although not shown, handle 101 may include one or more biasing elements (e.g., one or more springs), for example, such that proximal movement of first movable body 106 relative to stationary body 104 is biased distally. In these aspects, when the user releases a proximal pressure or force on first movable body 106, the one or more biasing elements may bias first movable body 106 distally. Alternatively, the one or more biasing elements (e.g., one or more springs) may bias first movable body 106 proximally. In these aspects, when the user releases a distal pressure or force on first movable body 106, the one or more biasing elements may bias first movable body 106 proximally.

First movable body 106 may include one or more reduced diameter sections or grooves 106A, for example, between a proximal ridge 106B and a distal ridge 106C, which may help to facilitate the user's grip of first movable body 106. For example, the user may place first movable body 106 between two fingers such that the user's fingers are positioned within groove(s) 106A. Accordingly, movement of the user's fingers (i.e., relative to the user's thumb in finger ring 112) controls the position of first movable body 106 relative to stationary body 104, and thus the movement of control element(s) 108 and/or one or more portion(s) of distal portion 102.

Stationary body 104 may include one or more visual or physical indicator(s) 116. Indicator(s) 116 may physically or visually indicate the actuation, or extension, of one or more portion(s) of distal portion 102. For example, as first movable body 106 is advanced distally within handle slot 114 relative to stationary body 104, thus actuating one or more portion(s) of distal portion 102, first movable body 106 may have to overcome, for example, indicator(s) 116. Indicator(s) 116 may signal (e.g., via a tactile signal or feedback, via a visual signal, etc.) to the user that distal portion 102 has been actuated, or is in an extended position. Indicator(s) 116 may be on one side of handle 101 or on two opposing sides of handle 101 (e.g., opposite of indicator(s) 116 as shown in FIG. 1A). Indicator(s) 116 may be a bump, a cantilevered protrusion, an indentation, a ridge, or any combination of the like. For example, indicator(s) 116 on one side of handle 101 may be a bump and indicator(s) 116 on an opposing side of handle 101 may be a cantilevered protrusion. Many other combinations are also contemplated. Additionally or alternatively, the user may utilize one or more markings on handle 101 (e.g., on stationary body 104 and/or first movable body 106) or other visual indicators to determine a status, position, actuation status, etc. of one or more portion(s) of distal portion 102.

Handle 101 may further include a second movable body, or a second actuator, 118. For example, movement of second movable body 118 may control the movement (e.g., rotation) of control element(s) 108, and thus one or more portion(s) of distal portion 102. For example, control element(s) 108 may be directly or indirectly coupled to second movable body 118 via, for example, an adhesive, a mechanical fit, a snap fit, a press fit, a weld (e.g., a laser weld), or any other coupling techniques commonly known in the art.

Second movable body 118 may be positioned on a distal portion of handle 101. For example, second movable body 118 may be positioned distally on stationary body 104 relative to first movable body 106. Additionally or alternatively, second movable body 118 may be between stationary body 104 and distal handle portion 111 of handle 101. Distal handle portion 111 and/or second movable body 118 may include one or more ridges or indentations, for example, to facilitate a user's grip on respective portion(s) of handle 101.

Distal handle portion 111 and/or second movable body 118 may, additionally or alternatively, include one or more visual or physical indicator(s), for example, two visual or physical indicators 120A and 120B. For example, indicator(s) 120A may be positioned on second movable body 118, and indicator(s) 120B may be on distal handle portion 111. Indicator(s) 120A and/or indicator(s) 120B may comprise one or more of the characteristics of indicator(s) 116, described above.

Indicator(s) 120A and indicator(s) 120B may identify or signal the amount of and/or direction of the rotational movement of a portion of distal portion 102, such as, for example, a helical coil 130. For example, a distance or a spacing between indicator(s) 120A on distal handle portion 111 and indicator(s) 120B on second movable body 118 may be indicative of the rotational movement of helical coil 130. For example, when indicator(s) 120A on distal handle portion 111 and indicator(s) 120B on second movable body 118 are aligned, helical coil 130 is not rotated. Similarly, when indicator(s) 120A on distal handle portion 111 and indicator(s) 120B on second movable body 118 are unaligned or misaligned, helical coil 130 is rotated (e.g., clockwise or counterclockwise). For example, clockwise rotation results in helical coil 130 being screwed into tissue, and counterclockwise rotation results in helical coil 130 being screwed out of tissue, or vice versa.

Although not shown, handle 101 may include one or more locks, for example, such that first movable body 106 and/or second movable body 118 may be locked in position. For example, indicator(s) 116 may include one or more indentation(s) and/or protrusion(s) such that, when first movable body 106 is advanced distally over indicator(s) 116, first movable body 106 is locked in position and/or unable to be retracted proximally without again overcoming indicator(s) 116. Second movable body 118 may additionally or alternatively include a lock or securing mechanism (not shown), for example, to prevent rotational movement of second movable body 118. The lock or securing mechanism may include, for example, a ratcheting mechanism having a ratchet and a pawl, or any other lock or securing mechanism commonly known in the art. The lock or securing mechanism may alternatively or additionally include one or more biasing features, for example, to assist with controlling the movement or rotation of first movable body 106 and/or second movable body 118.

Although not shown, in some aspects, handle 101 may not include first movable body 106 and/or second movable body 118. In such an embodiment, medical device 100 may not include external sheath 110. Accordingly, control element(s) 108 may be fixedly coupled (e.g., directly or indirectly) to handle 101. In this configuration, distal portion 102 may be rotated by rotating handle 101, for example, in its entirety. Rotation of handle 101 then rotates control element(s) 108 and distal portion 102, including helical coil 130. For example, clockwise rotation of handle 101 results in helical coil 130 being screwed into tissue, and counterclockwise rotation of handle 101 results in helical coil 130 being screwed out of the tissue, or vice versa.

Distal portion 102, shown in more detail in circle A of FIG. 1A, includes a detachable portion 124 and a coupler 126. Detachable portion 124 is configured to be coupled, or attached, and/or uncoupled, or detached, from a distal portion 108D of control element(s) 108, for example, via coupler 126. Coupler 126 is configured such that detachable portion 124 may be coupled and/or uncoupled from control element(s) 108 one or more times, for example, throughout a procedure. Accordingly, in some aspects, detachable portion 124 may be used two or more times throughout the procedure.

In alternative configurations, a procedure may require two or more detachable portions 124. In such a configuration, two or more detachable portions 124 may be used with the same coupler 126. For example, a first detachable portion 124 may be coupled to coupler 126. Once the first detachable portion 124 is coupled to the target tissue and uncoupled from coupler 126, a second detachable portion 124 may then be coupled to coupler 126. Accordingly, multiple detachable portions 124 may be used with the same coupler 126. In such a way, medical device 100 may be reloadable.

Detachable portion 124 may have a cross-sectional diameter greater, smaller, or equal to a cross-sectional diameter of control element(s) 108. For example, detachable portion 124 may have a cross-sectional diameter between 0.05 inches and 0.25 inches, for example, between 0.10 inches and 0.21 inches. Similarly, control element(s) 108 may have a cross-sectional diameter between 0.05 inches and 0.25 inches, for example, between 0.10 inches and 0.21 inches. Accordingly, in some configurations, control element(s) 108 has/have a cross-sectional diameter of approximately 0.10 inches and detachable portion 124 has a cross-sectional diameter of approximately 0.05 inches, or vice versa. Control element(s) 108 and detachable portion 124 may alternatively have the same cross-sectional diameter.

A cross-section of control element(s) 108 may be circular (as shown), ovular, elliptical, square, rectangular, hexagonal, or any other shape commonly used in the art. Similarly, a cross-section of coupler 126 may be circular, semi-circular, ovular, elliptical, square, rectangular, hexagonal, or any other shape commonly used in the art. Control element(s) 108 and coupler 126 may have the same cross-sectional shape or may have different cross-sectional shapes. For example, control element(s) 108 may have a circular cross-sectional shape, and coupler 126 may have a rectangular or square cross-sectional shape. Many other combinations are also contemplated.

Furthermore, a cross-section of detachable portion 124 may be circular (as shown), ovular, elliptical, square, rectangular, hexagonal, or any other shape commonly used in the art. Detachable portion 124 may have a same or similar cross-sectional shape as compared to control element(s) 108 and/or coupler 126. For example, detachable portion 124, coupler 126, and/or control element(s) 108 may each have a circular cross-sectional shape. Alternatively, detachable portion 124 may have a hexagonal cross-sectional shape, coupler 126 may have a rectangular cross-sectional shape, and/or control element(s) 108 may have a circular cross-sectional shape. Many other combinations are also contemplated. As shown in FIGS. 1A and 1B, a first portion 126A of coupler 126 extends distally from distal portion 108D of control element(s) 108. First portion 126A may be coupled (directly or indirectly) to distal portion 108D of control element(s) 108. For example, first portion 126A may be coupled to distal portion 108D of control element(s) 108 by, for example, an adhesive, a weld (e.g., a laser weld), a swage, a crimp, or any other coupling techniques commonly known in the art. In alternative configurations, first portion 126A is integrally formed with distal portion 108D of control element(s) 108. First portion 126A and second portion 126B may be detached or uncoupled, as described above. Accordingly, detachable portion 124 may be coupled to and/or uncoupled from control element(s) 108.

Although not shown, in further alternative configurations, first portion 126A of coupler 126 may be coupled to one or more secondary control element(s) confined or otherwise positioned within control element(s) 108. In such a configuration, control element(s) 108 may each be a sheath having a lumen. The secondary control element(s) (not shown) may be contained or otherwise positioned within the lumen of control element(s) 108. Accordingly, the secondary control element(s) may be configured to be independently movable relative to control element(s) 108. In such a configuration, first portion 126A and second portion 126B may be detached or uncoupled, as described above. In such a way, detachable portion 124 may be coupled to and/or uncoupled from the secondary control element(s) (not shown).

As shown in FIG. 1B, a cross-section (e.g., perpendicular to the longitudinal axis of medical device 100 shown in FIG. 1A) of first portion 126A and/or second portion 126B may be rectangular, triangular, semi-circular, semi-elliptical, circular, ovular, or any other shape commonly used in the art. In some configurations, a cross-section of first portion 126A and second portion 126B may be similarly shaped or differently shaped. For example, a cross-section of each of first portion 126A and second portion 126B may be semi-circular or rectangular. Alternatively, a cross-section of first portion 126A may be semi-circular, and a cross-section of second portion 126B may be rectangular.

A second portion 126B of coupler 126 extends proximally from a proximal portion 124A of detachable portion 124. Second portion 126B may be coupled (directly or indirectly) to a proximal portion 124A of detachable portion 124. For example, second portion 126B may be coupled to proximal portion 124A of detachable portion 124 by, for example, an adhesive, a weld (e.g., a laser weld), a swage, a crimp, or any other coupling techniques commonly known in the art. In alternative configurations, second portion 126B is integrally formed with proximal portion 124A of detachable portion 124.

In these aspects, first portion 126A and second portion 126B may help to maintain a connection between control element(s) 108 and detachable portion 124. First portion 126A and second portion 126B also allow for a user to separate first portion 126A and second portion 126B, thus separating control element(s) 108 from detachable portion 124. For example, first portion 126A and second portion 126B may be separated by pulling first portion 126A and second portion 126B apart with sufficient force to overcome a coupling force between first portion 126A and second portion 126B.

First portion 126A and second portion 126B may be detachably coupled. For example, first portion 126A may comprise a magnet, one or more magnetic materials (e.g., steel, iron, cobalt, nickel, neodymium, etc.) or magnetic alloys, and second portion 126B may comprise one or more magnetic materials (e.g., steel, iron, cobalt, nickel, neodymium, etc.) or magnetic alloys. In these aspects, first portion 126A and second portion 126B are of opposite polarity. In further embodiments, the magnet, magnetic material(s), or magnetic alloy(s) comprising first portion 126A and/or the magnetic material(s) or magnetic alloy(s) comprising second portion 126B may be surrounded (e.g., partially or completely encapsulated) by an insulating material. The insulating material may assist in preventing the magnet or one or more magnetic materials or magnetic alloys from coupling to undesired components. For example, the insulating material may assist in preventing the magnet or one or more magnetic materials or magnetic alloys from coupling to other medical devices or tools, external magnets, external magnetic materials, etc.

The insulating material partially or completely surrounding the magnet, magnetic material(s), or magnetic alloy(s) comprising first portion 126A and/or the magnetic material(s) or magnetic alloy(s) comprising second portion 126B may assist in facilitating a localized coupling joint between first portion 126A and second portion 126B. For example, partially or completely surrounding the magnet, magnetic material(s), or magnetic alloy(s) comprising first portion 126A and/or the magnetic material(s) or magnetic alloy(s) comprising second portion 126B may help to ensure first portion 126A and second portion 126B are coupled in the same orientation each time first portion 126A and second portion 126B are coupled.

First portion 126A and second portion 126B may additionally or alternatively be detachably coupled by other mechanisms. For example first portion 126A and second portion 126B may detachably couple via a latch, a thread, a bayonet mount, a snap-fit, a slide-fit, a releasable adhesive, a friction fit, or any combination of the like. First portion 126A and second portion 126B may additionally or alternatively include one or more keyed features such as, for example, slot(s), notch(es), or any combination of the like. Accordingly, the one or more keyed features may allow for first portion 126A and second portion 126B to be securely coupled and/or such that first portion 126A and second portion 126B may be repeatedly coupled or uncoupled in the same manner.

First portion 126A and second portion 126B may also be uncoupled by, for example, breaking, tearing, or otherwise separating first portion 126A from second portion 126B. For example, although not shown, detachable portion 124 may include one or more tabs and/or one or more perforations between first portion 126A and second portion 126B. Accordingly, first portion 126A and second portion 126B may be separated by breaking the one or more tab and/or tearing the one or more perforations.

Additionally or alternatively, first portion 126A and second portion 126B may be coupled by an adhesive or a material that deteriorates when the adhesive or material is exposed to the patient's gastrointestinal tract. The adhesive material may be positioned on a portion 127 of surface 126E of first portion 126A of coupler 126 that contacts or abuts surface 126F of second portion 126B. Portion 127 may comprise a portion or an entirety of surface 126E of first portion 126A. Although not shown, surface 126F of second portion 126B of coupler 126 may, additionally or alternatively, include a similar portion on surface 126F of second portion 126B that contacts or abuts surface 126E of first portion 126A. For example, the adhesive or material may deteriorate, or degrade, because of exposure to the moisture, humidity, temperature, etc., of the body. The adhesive or material may begin to deteriorate, for example, when portion 127 is at least partially uncovered by external sheath 110. The adhesive or material may deteriorate, for example, over a period of time to allow for positioning of distal portion 102 at or near the treatment site. For example, distal portion 102, including first portion 126A and second portion 126B, may be at least partially covered (e.g., by external sheath 110) during delivery of distal portion 102 to the treatment site. Because distal portion 102 is at least partially covered by external sheath 110, first portion 126A and second portion 126B may be shielded from the elements of the body such that deterioration of the adhesive or material is prevented or slowed.

Once a desired position is reached, distal portion 102 may be extended via first movable body 106 of handle 101. Distal portion 102 may be extended such that first portion 126A and second portion 126B are exposed to the moisture, humidity, temperature, etc., of the body and such that the adhesive or material begins to break down or deteriorate. In such a way, first portion 126A and second portion 126B may be uncoupled.

Additionally or alternatively, first portion 126A and second portion 126B may be coupled via electromagnetic principles. For example, first portion 126A may include an electromagnet, and second portion 126B may be comprised of a magnetic material. Accordingly, first portion 126A may be magnetized and demagnetized to be selectively coupled to and/or uncoupled from second portion 126B. In this configuration, control element(s) 108 may include one or more lumens (not shown) to accommodate wire(s) or cable(s) necessary to power the electromagnet of first portion 126A. Additionally, handle 101 may include one or more ports or plugs (not shown), such that medical device 100 may be coupled to a power source, for example, to provide energy to the electromagnet of first portion 126A. In alternative embodiments, handle 101 comprises an internal power source configured to magnetize and/or demagnetize the electromagnet of first portion 126A.

In some configurations, first portion 126A and second portion 126B may at least partially overlap when coupled, as shown in FIG. 1A. For example, a distal face 126C of first portion 126A may be distal of a proximal face 126D of second portion 126B, and proximal face 126D of second portion 126B may be proximal to distal face 126C of first portion 126A. In alternative configurations, a distal face of first portion 126A and a proximal face of second portion 126B are removably coupled such that no part of first portion 126A and second portion 126B overlap.

First portion 126A may be at least partially complementarily shaped according to second portion 126B, or vice versa. For example, a surface 126F of second portion 126B may be concave. Accordingly, a surface 126E of first portion 126A that contacts or abuts surface 126F of second portion 126B may be convex, for example, such that surface 126E of first portion 126A is partially or completely in contact with surface 126F of second portion 126B. Similarly, surface 126F of second portion 126B may include one or more protrusions, projections, ridges, indentations, cavities, etc. Accordingly, surface 126E of first portion 126A may be complementarily shaped such, for example, that surface 126E of first portion 126A is partially or completely in contact with surface 126F of second portion 126B.

A loop 128 is coupled to detachable portion 124. For example, at least a portion of loop 128 may be positioned proximal to a distalmost portion 124C of detachable portion 124 and distal to proximal portion 124A of detachable portion 124. A first portion 128A of loop 128 may at least partially extend around an external surface of detachable portion 124.

First portion 128A of loop 128 may be coupled to the external surface of detachable portion 124 via, for example, an adhesive, a weld (e.g., a laser weld), a swage, a crimp, or any other coupling techniques commonly known in the art. Additionally or alternatively, the external surface of detachable portion 124 may include a groove (not shown) such that first portion 128A of loop 128 may be coupled to the external surface of detachable portion 124 by, for example, a mechanical fit, a snap fit, a press fit, or any other coupling techniques commonly known in the art. In some embodiments, first portion 128A of loop 128 may be comprised of one or more resilient material(s) such as, for example, elastic polymeric or rubber materials, or any other material commonly used in the art. First portion 128A of loop 128 may additionally or alternatively be comprised of Teflon® or nylon filaments (e.g., similar to dental floss) or one or more suture materials (e.g., surgical steel, silk, cotton, and/or linen).

In alternative embodiments, first portion 128A of loop 128 is comprised of one or more rigid or semi-rigid material(s) such as, for example, metals, polymers, metal-polymer composites, and the like. In some embodiments, first portion 128A of loop 128 forms a continuous loop. In alternative embodiments (not shown), first portion 128A is C-shape, or forms a discontinuous loop. A cross-section of first portion 128A of loop 128 may be circular, ovular, elliptical, square, rectangular, hexagonal, or any other shape commonly used in the art.

A second portion 128B of loop 128 may extend radially outward from detachable portion 124. In some embodiments, second portion 128B forms a continuous loop. Second portion 128B of loop 128 may be comprised of one or more resilient material(s) such as, for example, elastic polymeric or rubber materials, or any other material commonly used in the art. Second portion 128B of loop 128 may additionally or alternatively be comprised of Teflon® or nylon filaments (e.g., similar to dental floss) or one or more suture materials (e.g., surgical steel, silk, cotton, and/or linen). Second portion 128B may be comprised of the same material as first portion 128A or of a different material. For example, second portion 128B may be comprised of a first rubber material, and first portion 128A may be comprised of a second rubber material, different from the first. Various other combinations of materials are also considered. A cross-section of second portion 128B may be circular, ovular, elliptical, square, rectangular, hexagonal, or any other shape commonly used in the art. In some configurations, second portion 128B is larger than first portion 128A. For example, second portion 128B may have a circumference and/or a diameter greater than first portion 128A.

Distal portion 102 further includes a helical coil 130. Helical coil 130 is configured to be coupled to and decoupled from tissue. Helical coil 130 may be at least partially disposed within a cavity, or lumen, 132 of detachable portion 124. At least a portion of helical coil 130 may be distal to distalmost end 124D of detachable portion 124. Helical coil 130 may be comprised of a plurality of windings coupled (directly or indirectly) to detachable portion 124. Helical coil 130 may be coupled (directly or indirectly) to detachable portion 124 via, for example, a weld (e.g., a laser weld), an adhesive, a mechanical fit, a snap fit, a crimp, a swage, or other mechanisms commonly known in the art. In alternative configurations, helical coil 130 is integrally formed with detachable portion 124. For example, a wire or coil forming helical coil 130 may also comprise or form detachable portion 124. In such a configuration, helical coil 130 forms at least a distalmost portion 124C of detachable portion 124.

Helical coil 130 may include, for example, one to ten, or three to five, full helical turns or windings. In some configurations, the plurality of windings forming helical coil 130 may be evenly spaced. In alternative configurations, a space between each of the plurality of windings forming helical coil 130 may vary. For example, in a proximal region 130A of helical coil 130, one or more adjacent windings may be spaced by a first distance. For example, the plurality of windings forming helical coil 130 in proximal region 130A may be in contact with one another. In a distal region 130C of helical coil 130, adjacent windings may be spaced by a second distance. In an intermediate region 130B, for example, between proximal region 130A and distal region 130C, adjacent windings may be spaced by a third distance. In some configurations, the second distance may be greater than the third distance, and the third distance may be greater than the first distance. Many other configurations are also contemplated. In further configurations, the second distance is equal to the third distance such that intermediate region 130B and distal region 130C are similarly spaced.

In further configurations, windings forming intermediate region 130B may have a non-constant pitch, resulting in a gradually increasing spacing. For example, the windings in intermediate region 130B may transition from adjacent windings being in contact with one another in proximal region 130A to the windings being spaced apart in distal region 130C.

Distal tip 130D of helical coil 130 may be narrowed or sharpened, for example, to more easily pierce or penetrate the tissue of the subject. In some embodiments, distal tip 130D of helical coil 130 is oriented in a bent or curved configuration, such that, for example, distal tip 130D of helical coil 130 is parallel or substantially parallel to the windings forming helical coil 130. In such a configuration, distal tip 130D may be less likely to pierce or puncture tissue surrounding the target area. Helical coil 130 may be formed from a solid round wire or a plurality of wires. In some configurations, helical coil 130 may be configured to have a diameter less than an inner diameter of detachable portion 124. For example, helical coil 130 may have an outer cross-sectional diameter between 0.002 inches and 0.200 inches, for example between 0.005 inches and 0.020 inches. Helical coil 130 may have an inner cross-sectional diameter between 0.001 inches and 0.190 inches, for example, between 0.002 inches and 0.015 inches. In some configurations, the wire(s) forming helical coil 130 may have a cross-sectional diameter greater than or equal to a wire or a plurality of wires forming detachable portion 124. In alternative configurations, the wire(s) forming helical coil 130 may have a cross-sectional diameter less than a wire or a plurality of wires forming detachable portion 124. For example, the wire(s) forming helical coil 130 may have a cross-sectional diameter between 0.0005 inches and 0.100 inches, for example, between 0.005 inches and 0.015 inches.

Although not shown, helical coil 130 may include one or more markers, such as, for example, radiopaque marker(s), ultrasonic marker(s), etc., to assist the user with determining a status, position, actuation status, etc. of helical coil 130. For example, a winding or portion of a winding in proximal region 130A and/or intermediate region 130B may include a marker to signal to the user how deep the helical coil 130 is within a tissue, how many windings of helical coil 130 has been inserted into the tissue, etc.

Referring still to FIG. 1A, control element(s) 108 may extend from one or more portions of handle 101 of medical device 100 (e.g., from a distal portion of first movable body 106) to one or more portions of distal portion 102. Control element(s) 108 may each be formed by a wire, a coil, a tube, etc. Additionally, external sheath 110 may extend from one or more portions of handle 101 (e.g., from a distal portion of distal handle portion 111) to one or more portions of distal portion 102. External sheath 110 may be formed by a coil, a braid, a tube, a sheath, etc., and lumen 113 may extend longitudinally within external sheath 110, for example, to receive portion(s) of control element(s) 108. In these aspects, control element(s) 108 may be radially within external sheath 110. For example, control element(s) 108 may be longitudinally and/or rotatably movable relative to external sheath 110 and at least partially disposed within lumen 113 of external sheath 110.

In some configurations, such as, for example, when medical device 100 is in a retracted or closed configuration, at least a portion of distal portion 102 is also disposed within lumen 113 of external sheath 110. For example, helical coil 130 may be within or positioned proximal to a distalmost end of 110A of external sheath 110. In such a configuration, first movable body 106 may be in a proximal position, for example, within handle slot 114.

In alternative configurations, medical device 100 may not include external sheath 110, first movable body 106, and/or second movable body 118. In such a configuration, medical device 100 may simply include a gripping portion for a user to grasp medical device 100, for example, a proximal portion of medical device 100, and distal portion 102 with detachable portion 124. Distal portion 102 may be directly or indirectly coupled to the gripping portion by, for example, control element(s) 108. In such aspects, helical coil 130 of detachable portion 124 may be driven into tissue by rotating the gripping portion, thus rotating control element(s) 108 and helical coil 130. Detachable portion 124 may be uncoupled by pulling or urging the gripping portion proximally, for example, to overcome any connective forces between detachable portion 124 and the remainder of medical device 100. In this configuration, medical device 100 may include fewer components, and thus may be easier and/or less costly to manufacture or produce.

FIG. 1B illustrates distal portion 102 in a second or detached configuration. For example, in the detached configuration, first portion 126A of coupler 126 is detached or uncoupled from second portion 126B of coupler 126. First portion 126A and second portion 126B may be detached or uncoupled as described above. Accordingly, detachable portion 124 is uncoupled or detached from control element(s) 108.

A cross-section (e.g., perpendicular to the longitudinal axis of medical device 100 shown in FIG. 1A) of first portion 126A and/or second portion 126B may be rectangular, triangular, semi-circular, semi-elliptical, circular, ovular, or any other shape commonly used in the art. In some configurations, a cross-section of first portion 126A and second portion 126B may be similarly shaped or differently shaped. For example, a cross-section of each of first portion 126A and second portion 126B may be semi-circular or rectangular. Alternatively, a cross-section of first portion 126A may be semi-circular, and a cross-section of second portion 126B may be rectangular.

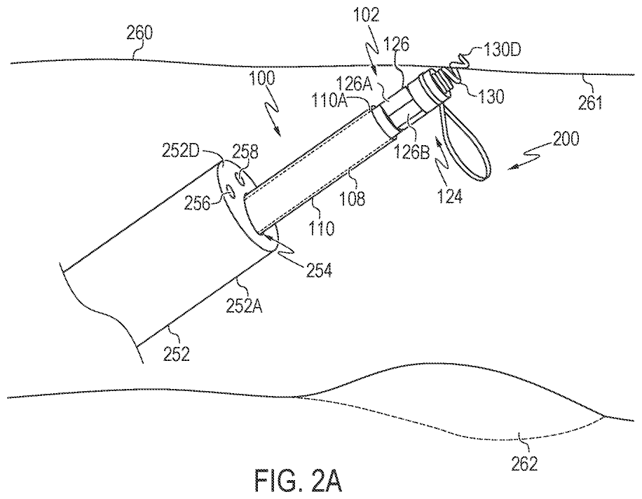

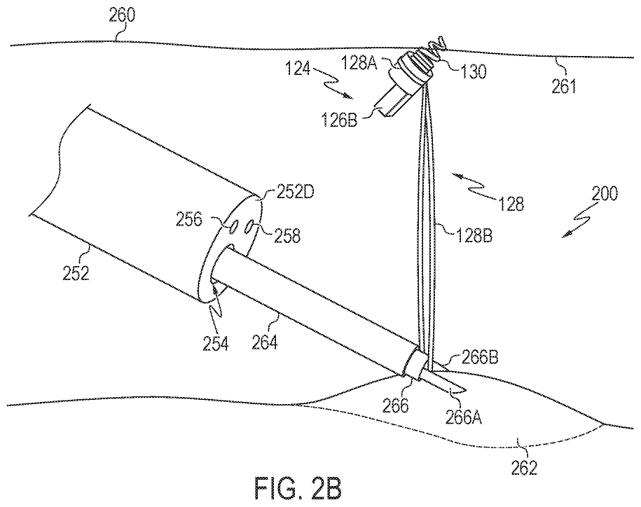

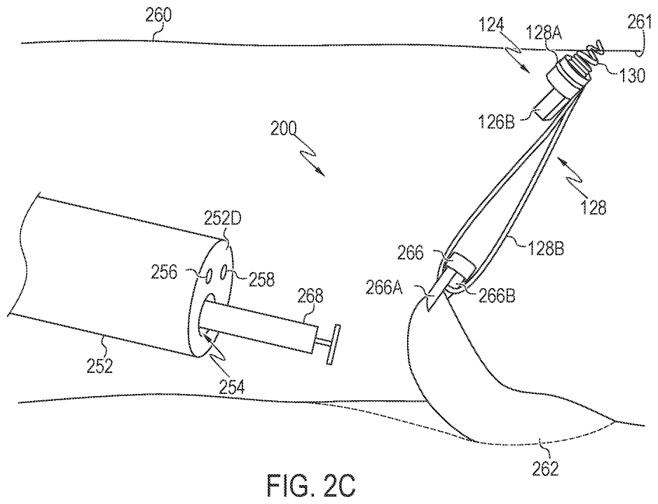

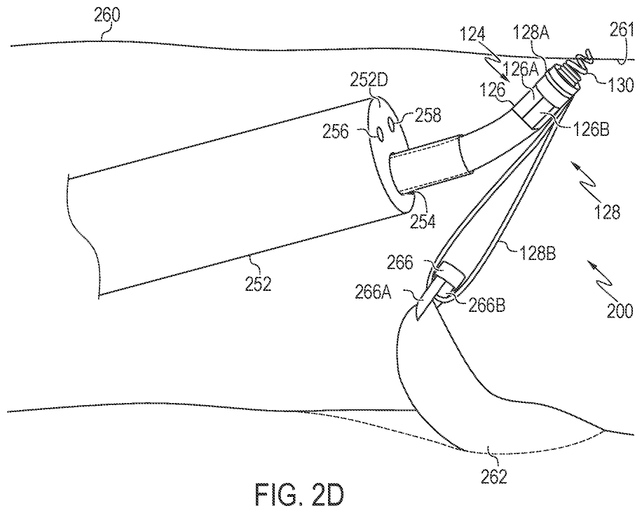

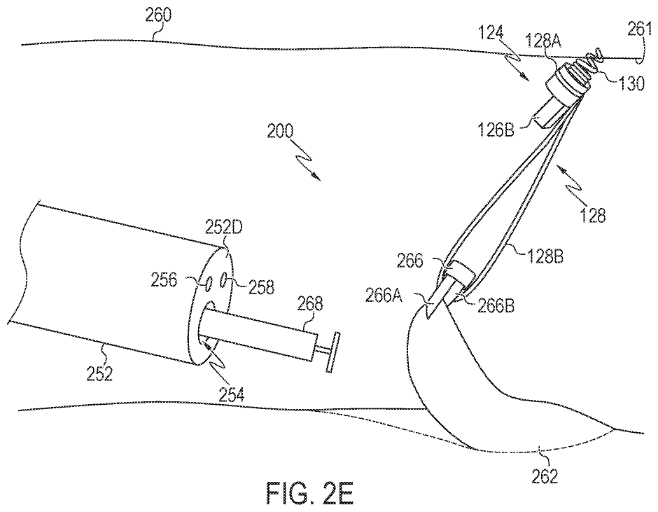

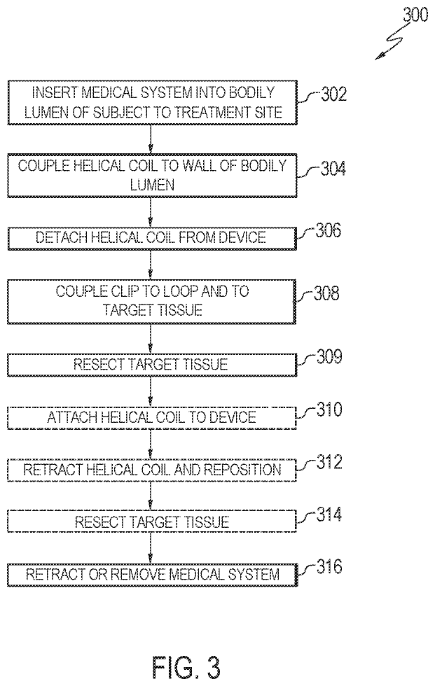

Referring now to FIG. 2A, a medical system 200 is shown. Medical system 200 includes medical device 100 (described above and shown in FIGS. 1A and 1B) and an endoscope 252. Medical device 100 may be disposed within a working channel, or lumen, 254 of endoscope 252. In alternative configurations (not shown), medical device 100 may extend externally to endoscope 252 (e.g., along an external surface of endoscope 252), for example, without extending through working channel 254. Alternatively, one or more portions of medical system 200 may be inserted via and/or surrounded via an insertion sheath (not shown), for example, surrounding endoscope 252. Additionally, FIG. 3 illustrates an exemplary method, or process, 300 that one or more users may perform with any of the medical systems, devices, or portions of systems discussed herein, for example, medical system 200.

Although not shown, one or more portions of endoscope 252 (e.g., a distal portion 252A) may be deflectable, for example, via one or more knobs, levers, buttons, or other controls on a proximal handle (not shown) of endoscope 252. In these aspects, distal portion 252A of endoscope 252 may be maneuvered while being delivered through a bodily lumen, to the treatment site and/or positioned relative to the treatment site, for example, in a retroflex position, which may be used when the treatment site is in the subject's esophagus, stomach, duodenum, colon, or other portion of the gastrointestinal tract. Accordingly, at least portions of medical device 100 may be flexible or semi-flexible, for example, in order to be maneuvered along with endoscope 252.

Additionally, a distal face 252D of endoscope 252 may include one or more visualization device(s) 256 (e.g., one or more cameras, light emitting diodes (LEDs), one or more image sensors, endoscope viewing elements, optical assemblies, including one or more image sensors and one or more lenses, etc.). Distal face 252D of endoscope 252 may, additionally or alternatively, include one or more opening(s) 258 configured to supply a liquid, a gas, and/or suction to the bodily lumen or treatment site.

In referring to FIGS. 2A and 3, in a step 302, medical system 200 (e.g., a distal portion of medical system 200) may be inserted into a bodily lumen 260 of the subject, for example, through a natural orifice or an incision, to treatment site 262. Before or after medical system 200 reaches treatment site 262, first movable body 106 (shown in FIG. 1A) may be actuated, extended, pushed, or urged distally within handle slot 114 of handle 101, such that control element(s) 108 is actuated, extended, pushed, or urged distally and, accordingly, such that at least a portion of distal portion 102 is distal to distalmost end 110A of external sheath 110.

Endoscope 252 and/or medical device 100 may then be maneuvered to position helical coil 130 of medical device 100 in contact with a wall 261 of lumen 260, for example, opposite of or across lumen 260 from treatment site 262. Endoscope 252 may be maneuvered by, for example, deflecting distal portion 252A, advancing endoscope 252 distally, and/or retracting endoscope 252 proximally. Additionally or alternatively, visualization device(s) 256 may be used to maneuver and/or reposition endoscope 252. Similarly, medical device 100 may be maneuvered by, for example, advancing medical device 100 distally, and/or retracting medical device 100 proximally.

Once helical coil 130 is in contact with wall 261 of lumen 260, control element(s) 108 and, thus, distal portion 102 and helical coil 130 may be rotated in a first direction (e.g., clockwise or counterclockwise). Control element(s) 108 may be rotated by, for example, rotating second movable body 118 (shown in FIG. 1A) in the first direction. Accordingly, as helical coil 130 is rotated in the first direction, distal tip 130D of helical coil 130 may pierce wall 261 of lumen 260. Continued rotation of helical coil 130 in the first direction may cause distal tip 130D to be driven deeper into the tissue of wall 261, for example, in a corkscrew manner.

Accordingly, as shown in FIG. 2A and described in a step 304, helical coil 130 may be anchored, or coupled, to wall 261 of lumen 260, for example, opposite of or across lumen 260 from treatment site 262. One or more windings of helical coil 130 may be embedded into wall 261 of lumen 260, for example, to ensure helical coil 130 is securely fastened, coupled, or attached to wall 261 of lumen 260. Markers described previously in FIG. 1A may be used to determine, for example, how many windings are or what portion of helical coil 130 is within wall 261 of lumen 260.

As shown in FIG. 2B, in a step 306, detachable portion 124 is then uncoupled or detached from control element(s) 108 (FIG. 1B). Detachable portion 124 may be uncoupled from control element(s) 108 by, for example, pulling or retracting control element(s) 108 such that the pulling or retracting force overcomes the connective forces between first portion 126A (shown in FIGS. 1A, 1B, and 2A) and second portion 126B of coupler 126. In such a way, detachable portion 124 remains attached, or coupled, to wall 261, while the remainder of medical device 100 is pulled proximally and/or retracted.

Still referring to FIG. 2B, medical system 200 may further include a device 264 having a clip 266. Additionally, medical system 200 may include a cutting tool 268 (shown in FIGS. 2C and 2E). Device 264 and cutting tool 268 are described in further detail below. Each of device 264 and cutting tool 268 may be configured to be disposed within working channel 254 of endoscope 252. For example, device 264 may be inserted into working channel 254 of endoscope 252. Before, during, or after device 264 is removed from working channel 254, cutting tool 268 may be inserted into working channel 254. As shown in FIGS. 2B-2E, clip 266 may be any clip commonly used in the art, for example, clip 266 may be a hemoclip or an endoclip. Clip 266 may include a first leg 266A and a second leg 266B.

Referring still to FIG. 2B, after detachable portion 124 of medical device 100 is anchored to wall 261 of lumen 260, device 264 may be inserted through working channel 254 of endoscope 252, such that a distal portion of device 264 is distal to distal face 252D of endoscope 252. A portion of second portion 128B of loop 128 may be positioned within clip 266, for example, between first leg 266A and second leg 266B. To position second portion 128B of loop 128 between first leg 266A and second leg 266B, endoscope 252 may be pushed distally, pulled proximally, rotated, or deflected within lumen 260, and/or device 264 may be pushed distally, pulled proximally, or rotated within working channel 254 of endoscope 252.

In some configurations, endoscope 252 may be oriented such that working channel 254 is positioned closer to second portion 128B, for example, to assist with positioning second portion 128B between first leg 266A and second leg 266B. For example, endoscope 252 may be orientated such that working channel 254 is in a 6 o'clock position relative to visualization device(s) 256 and/or opening(s) 258, as shown. In alternative configurations, endoscope 252 may be oriented such that working channel 254 is in a 3 o'clock, 9 o'clock, or 12 o'clock position relative to visualization device(s) 256 and/or opening(s) 258. In such a way, endoscope 252 may be rotated such that working channel 254 is appropriately positioned and/or oriented to couple device 264 to second portion 128B of loop 128. Many other configurations are also contemplated, for example, to assist with positioning second portion 128B between first leg 266A and second leg 266B of clip 266.

Referring to FIGS. 2B and 3, in a step 308, clip 266 may be coupled, or anchored, to treatment site 262. Clip 266 is subsequently released from the remainder of device 264. Accordingly, second portion 128B of loop 128 may be anchored to treatment site 262, for example, between clip 266 and treatment site 262. In such a manner, second portion 128B may be anchored between first leg 266A and second leg 266B of clip 266 and to treatment site 262. With detachable portion 124 anchored to wall 261 of lumen 260 and second portion 128B of loop 128 anchored to treatment site 262, second portion 128B of loop 128 may be subjected to tension, and tissue of treatment site 262 may be at least partially lifted or similarly tensioned.

As shown in FIGS. 20, once device 264 is removed from working channel 254, cutting tool 268, for example, a knife, scissors, a blade, an electrocautery knife, or any other resection or cutting device may be introduced through working channel 254 of endoscope 252. In a step 309, cutting tool 268 may be used to at least partially cut or resect tissue from treatment site 262. The tension of second portion 128B may help cause the resected tissue of treatment site 262 to lift, for example, as treatment site 262 is cut or resected. Accordingly, as the resected tissue of treatment site 262 is lifted, the tension of second portion 128B may decrease, and second portion 128B may revert back to its original position.

As shown in FIG. 2D and described in an optional step 310, detachable portion 124 may be, once again, coupled, or attached, to medical device 100, as described below. Subsequently, in an optional step 312, helical coil 130 may be repositioned within lumen 260. Accordingly, second portion 128B of loop 128 may be re-tensioned, for example, by repositioning helical coil 130, for example, relative to treatment site 262.

To reposition helical coil 130 and, subsequently, to re-tension second portion 128B, medical device 100 (without detachable portion 124) may be re-inserted through working channel 254 of endoscope 252. Medical device 100 may be re-inserted through working channel 254 of endoscope 252, for example, once cutting tool 268 is removed from working channel 254 of endoscope 252. Endoscope 252 may be used to maneuver medical device 100 such that the first portion 126A of coupler 126 contacts second portion 126B of coupler 126. First portion 126A and second portion 126B may then be re-coupled or re-attached according to the attachment mechanisms described above. In such a manner, detachable portion 124 may be attached or coupled to control element(s) 108.

Once detachable portion 124 is coupled to control element(s) 108, control element(s) 108 may then be rotated in a second direction (e.g., counterclockwise or clockwise), opposite the first direction. Accordingly, as control element(s) 108 is/are rotated in the second direction, helical coil 130 is also rotated in the second direction, for example, such that helical coil 130 may be uncoupled, or withdrawn, from wall 261 of lumen 260.

Once uncoupled or withdrawn from wall 261 of lumen 260, detachable portion 124 and helical coil 130 may be repositioned, for example, to a position distal of treatment site 262, such that second portion 128B of loop 128 is, once again, under tension. For example, endoscope 252 and/or medical device 100 may then be maneuvered to reposition helical coil 130 of medical device 100 in contact with wall 261 of lumen 260, for example, opposite of or across lumen 260 from treatment site 262. Endoscope 252 may be maneuvered by, for example, deflecting distal portion 252A, advancing endoscope 252 distally, and/or retracting endoscope 252 proximally. Medical device 100, including detachable portion 124 and helical coil 130, may also be maneuvered by, for example, advancing medical device 100 distally, and/or retracting medical device 100 proximally.

Throughout optional step 310 and optional step 312, second portion 128B of loop 128 may remain anchored to treatment site 262, for example, between first leg 266A and second leg 266B of clip 266.

As shown in FIG. 2E, detachable portion 124 may, once again, be detached or uncoupled from control element(s) 108, as described above. For example, once medical device 100 is removed from working channel 254 of endoscope 252, cutting tool 268 may be re-introduced, for example, through working channel 254 of endoscope 252. Cutting tool 268 may then be used to continue to cut or resect tissue from treatment site 262 in an optional step 314. Optional step 310, optional step 312 and/or optional step 314 may be repeated such that helical coil 130 may be repositioned and/or second portion 128B of loop 128 may be re-tensioned numerous times, for example, until treatment site 262 is resected to the desired amount. Additionally or alternatively, optional step 310 and optional step 312 may be performed before resecting tissue from treatment site 262 in step 309, for example, if there is not the desired tension in second portion 128B or otherwise improper positioning of detachable portion 124 after helical coil 130 is coupled to wall 261 of lumen 260.

Once the tissue from treatment site 262 is appropriately resected in step 309 or optional step 314, in a step 316, detachable portion 124, and thus helical coil 130, may be detached from wall 261, as described above. For example, once cutting tool 268 is removed from working channel 254 of endoscope 252, medical device 100 may be re-introduced through working channel 254 of endoscope 252. Medical device 100 may be maneuvered (e.g., via manipulating medical device 100 and/or endoscope 252) such that the first portion 126A of coupler 126 once again contacts second portion 126B of coupler 126. First portion 126A and second portion 126B may then be re-coupled or re-attached according to the attachment mechanisms described above. In such a manner, detachable portion 124 may be re-attached or re-coupled to control element(s) 108 and may be retracted or removed proximally. One or more portions of medical system 200 may then be retracted or removed proximally. For example, medical device 100, cutting tool 268, and/or endoscope 252 may be retracted or removed proximally. Accordingly, medical device 100 and/or the resected tissue from treatment site 262 coupled to clip 266 may then be removed from lumen 260.

In alternative embodiments, helical coil 130 may be biodegradable. For example, the wire(s) forming helical coil 130 may disintegrate or decompose such that detachable portion 124 detaches from wall 261. Detachable portion 124 may subsequently be removed using a basket, grasper, net, cage, suction, or other grasping tools or methods. Alternatively, detachable portion 124 may pass naturally by the subject. In such a manner, detachable portion 124 may detach from wall 261 without needing to re-couple or re-attach detachable portion 124 to medical device 100.

In some embodiments, cutting tool 268 is used to detach second portion 128B of loop 128 from medical device 100, such that, for example, resected tissue from treatment site 262 may be passed naturally by the subject or removed through working channel 254 of endoscope 252, for example, by applying suction. Although not shown, in some aspects, resected tissue from treatment site 262 may be removed through working channel 254 of endoscope 252, for example, by using one or more graspers, nets, cages, suction, or any other device(s) or method(s) commonly used in the art to remove objects or material from a subject.

Each embodiment discussed herein may enable the user to apply tension to tissue, for example, in order to lift tissue such that the tissue may be more easily cut, resected, removed, or otherwise treated. Each embodiment may help to decrease procedure time, a likelihood of patient injury, and/or a likelihood of other procedural complications.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

We claim:

1. A medical system, comprising:
an insertion device having a working channel;
a first medical device configured to be disposed within the working channel of the insertion device, the first medical device having:
a handle;
a control element extending from the handle;
a detachable portion, wherein the detachable portion is removably coupled to a distal portion of the control element;
a helical coil coupled to the detachable portion;
a loop, wherein a first portion of the loop is coupled to the detachable portion, and wherein a second portion of the loop extends radially away from the detachable portion; and
a second medical device configured to be disposed within the working channel of the insertion device, wherein the second medical device is configured to receive the second portion of the loop, and wherein the second medical device is configured to couple the second portion of the loop to a portion of a treatment site.

2. The medical system of claim 1, wherein the first medical device further includes a coupler, wherein the coupler includes a first portion coupled to or part of the distal portion of the control element, wherein the coupler includes a second portion coupled to or part of a proximal portion of the detachable portion, and wherein the first portion and the second portion are configured to releasably couple the detachable portion to the control element.

3. The medical system of claim 2, wherein the first portion of the coupler is configured to overlap with at least a portion of the second portion of the coupler.

4. The medical system of claim 2, wherein a first contacting surface of the first portion abuts a second contacting surface of the second portion, and wherein the first contacting surface of the first portion is complementarily shaped to the second contacting surface of the second portion.

5. The medical system of claim 2, wherein a portion of the first portion of the coupler is magnetic, and wherein a portion of the second portion of the coupler is magnetic of an opposite polarity of the portion of the first portion.

6. The medical system of claim 2, wherein a portion of the first portion of the coupler comprises an electromagnet, and wherein a portion of the second portion of the coupler is a magnetic material.

7. The medical system of claim 1, wherein rotation of the helical coil in a first direction is configured to couple the distal portion to a tissue wall, and wherein rotation of the helical coil in a second direction is configured to uncoupled the helical coil from the tissue wall.

8. The medical system of claim 1, wherein the helical coil includes a plurality of windings, wherein adjacent windings of a first portion of the plurality of windings are spaced by a first distance, wherein adjacent windings of a second portion of the plurality of windings are spaced by a second distance, and wherein adjacent windings of a third portion of the plurality of windings are spaced by a third distance.

9. The medical system of claim 8, wherein the third distance is greater than the second distance, and wherein the second distance is greater than the first distance.

10. The medical system of claim 1, wherein the handle includes a stationary body, a first movable body, and a second movable body, wherein the first movable body controls axial movement of the distal portion of the control element, wherein the second movable body controls rotational movement of the distal portion of the control element.

11. The medical system of claim 10, wherein the stationary body includes a first indicator to identify axial movement of the helical coil, and wherein the second movable body includes a second indicator to identify the rotational movement of the helical coil.

12. The medical system of claim 1, wherein the second medical device includes a clip.

13. The medical system of claim 1, wherein the second portion of the loop is comprised of a resilient material.

14. The medical system of claim 1, wherein the medical system includes a third medical device configured to be disposed within the working channel of the insertion device, wherein the third medical device is configured to cut tissue.

15. The medical system of claim 2, wherein the first portion of the coupler and the second portion of the coupler are coupled by an adhesive material,
wherein, in a first configuration, the adhesive material is disposed within a sheath extending from the handle; and
wherein, in a second configuration, the adhesive material is uncovered by the sheath,
wherein, in the second configuration, the adhesive material degrades to uncouple the first portion of the coupler and the second portion of the coupler.

16. A medical device, comprising:
a handle;
a control element extending from the handle;
a detachable portion, wherein the detachable portion is removably coupled to a distal portion of the control element;
a helical coil, coupled to the detachable portion; and
a loop having a first portion and a second portion, wherein the first portion of the loop is coupled to the detachable portion, and wherein the second portion of the loop extends radially outward from the detachable portion.

17. The medical device of claim 16, wherein the medical device further includes a coupler, wherein the coupler includes a first portion coupled to or part of the distal portion of the control element, wherein the coupler includes a second portion coupled to or part of a proximal portion of the detachable portion, and wherein the first portion and the second portion are configured to releasably couple the detachable portion to the control element.

18. The medical device of claim 17, wherein a portion the first portion of the coupler is a magnet, and wherein a portion of the second portion of the coupler is magnetic.

19. A method, comprising:
delivering an insertion device and a first medical device into a lumen to a treatment site, wherein the first medical device is configured to be disposed within a working channel of the insertion device, the first medical device comprising:
a handle;
a control element extending from the handle;
a detachable portion, wherein the detachable portion is removably coupled to a distal portion of the control element;
a helical coil coupled to the detachable portion; and
a loop, wherein a first portion of the loop is coupled to the detachable portion;
coupling the helical coil to a wall of the lumen;
uncoupling the detachable portion from the control element;
delivering a second medical device, wherein the second medical device is configured to be disposed within the working channel of the insertion device, wherein the second medical device is configured to receive a second portion of the loop, and wherein the second medical device includes a detachable clip;
attaching the detachable clip to the loop;
coupling the detachable clip to the treatment site;
delivering a third medical device, wherein the third medical device is configured to be disposed within the working channel of the insertion device; and
resecting at least a portion of the treatment site using the third medical device.

20. The method of claim 19, wherein, after delivering the third medical device and partially resecting the treatment site with the third medical device, the method further comprises:
delivering the first medical device;
coupling the first medical device to the detachable portion, including the helical coil;
repositioning the detachable portion;
delivering the third medical device; and
resecting at least a second portion of the treatment site using the third medical device.
